(19) United States
(12) Patent Application Publication
(10) Pub. No.: US 2006/0216263 A1
(43) Pub. Date: Sep. 28, 2006

(54) Polyvinylpyrrolidone composition and processes for its production

(76) Inventors: Takashi Miyai, Takatsuki-shi (JP); Mitsuru Nakajima, Takatsuki-shi (JP); Akio Naka, Suita-shi (JP)

Correspondence Address: WENDEROTH, LIND & PONACK, L.L.P., 2033 K STREET N. W., SUITE 800, WASHINGTON, DC, 20006-1021, US

(21) Appl. No.: 11/377,422

(22) Filed: Mar. 17, 2006

(30) Foreign Application Priority Data
Mar. 28, 2005 (JP) 2005-92132 (PAT.)

(51) Int. Cl. A61K 8/81

(52) U.S. Cl. 424/70.15

(57) ABSTRACT

A polyvinylpyrrolidone composition in a form of a solid preparation or an aqueous solution, the composition including polyvinylpyrrolidone, ammonia, and a secondary amine, and processes for producing the polyvinylpyrrolidone composition, the first process including heat drying a polyvinylpyrrolidone aqueous solution containing ammonia and a secondary amine to obtain the polyvinylpyrrolidone in a form of a solid preparation, the second process including adding a secondary amine to an aqueous solution containing polyvinylpyrrolidone and ammonia to obtain the polyvinylpyrrolidone composition in a form of an aqueous solution, and the third process including polymerizing N-vinyl-2-pyrrolidone using hydrogen peroxide as a polymerization initiator in a presence of a metal catalyst using ammonia as a promoter in an aqueous medium to obtain the polyvinylpyrrolidone composition in a form of an aqueous solution.

### BACKGROUNDS OF THE INVENTION

1. Field of the Invention

The present invention relates to a polyvinylpyrrolidone composition and processes for its production.

2. Description of the Prior Art

Polyvinylpyrrolidone has been used in a wide range of areas, for example, as a harmless functional polymer, for applications such as cosmetics, intermediates of drugs and agricultural chemicals, food additives, photosensitive electronic materials, and adhesion providing agents, or for various special industrial applications. Polyvinylpyrrolidone is usually produced by polymerizing N-vinyl-2-pyrrolidone using hydrogen peroxide as a polymerization initiator in a presence of a metal catalyst in an aqueous medium (e.g., see Japanese Patent Laid-open Publications No. 62-62804, No. 11-71414, and No. 2002-155108). The use of a primary, secondary, or tertiary amine as a promoter in the polymerization makes the rate of polymerization slow and brings the coloration of the resulting polymer (e.g., see Japanese Patent Laid-open Publication No. 62-62804). In contrast, the use of ammonia as a promoter in the polymerization allows the polymerization to rapidly progress and to prevent the coloration of the resulting polymer.

When produced by the process as described above, polyvinylpyrrolidone is obtained in a form of an aqueous solution. However, depending on its applications, polyvinylpyrrolidone is needed to be in a form of a solid preparation. A solid preparation of polyvinylpyrrolidone is obtained by heat drying an aqueous solution of polyvinylpyrrolidone to sufficiently evaporate the water content of the aqueous solution. However, if ammonia is contained in an aqueous solution of polyvinylpyrrolidone, the cross-linking reaction or graft reaction of polyvinylpyrrolidone may progress in the step of heat drying, resulting in a high molecular weight polyvinylpyrrolidone which is not soluble in water. Moreover, when the resulting solid preparation of polyvinylpyrrolidone is dissolved in water, the solution may cause gelation. Therefore, there has been a demand for high-quality polyvinylpyrrolidone which caused less coloration and substantially no formation of insoluble matter and gelled matter.

### SUMMARY OF THE INVENTION

Under these circumstances, an objective of the present invention is to provide high-quality polyvinylpyrrolidone as a composition in a form of a solid preparation or an aqueous solution, that is, to provide a polyvinylpyrrolidone composition which may cause substantially no formation of insoluble matter or gelled matter, even if dissolved in an aqueous medium, so that it has excellent solubility and causes less coloration, when it is in a form of a solid preparation; or which may cause substantially no formation of insoluble matter, even if heat dried, so that it has excellent heat resistance and low hue values, when it is in a form of an aqueous solution; as well as processes for producing such a composition in a simple and easy manner.

The present inventors have extensively studied and, as a result, have found that a solid preparation of polyvinylpyrrolidone containing substantially no insoluble matter can be obtained by allowing a secondary amine to coexist when an aqueous solution containing polyvinylpyrrolidone and ammonia is heat dried, and that the resulting solid preparation causes no gelation when dissolved in water, thereby completing the present invention.

Thus, the present invention provides a polyvinylpyrrolidone composition in a form of a solid preparation or an aqueous solution, the composition comprising polyvinylpyrrolidone, ammonia, and a secondary amine.

In the polyvinylpyrrolidone composition of the present invention, the secondary amine may preferably be selected from the group consisting of dialkanolamines and dialkylamines, and the most preferred secondary amine may be diethanolamine.

The present invention further provides three processes for producing a polyvinylpyrrolidone composition as described above.

The first process comprises heat drying a polyvinylpyrrolidone aqueous solution containing ammonia and a secondary amine to obtain the polyvinylpyrrolidone composition in a form of a solid preparation.

In the first process, the polyvinylpyrrolidone aqueous solution may preferably be obtained by polymerizing N-vinyl-2-pyrrolidone using hydrogen peroxide as a polymerization initiator in a presence of a metal catalyst using ammonia and the secondary amine as promoters in an aqueous medium.

In the first process, the polyvinylpyrrolidone aqueous solution may preferably be obtained by adding the secondary amine to an aqueous solution containing polyvinylpyrrolidone and ammonia. The aqueous solution containing polyvinylpyrrolidone and ammonia may more preferably be obtained by polymerizing N-vinyl-2-pyrrolidone using hydrogen peroxide as a polymerization initiator in a presence of a metal catalyst using ammonia as a promoter in an aqueous medium.

The second process comprises adding a secondary amine to an aqueous solution containing polyvinylpyrrolidone and ammonia to obtain the polyvinylpyrrolidone composition in a form of an aqueous solution.

In the second process, the aqueous solution containing polyvinylpyrrolidone and ammonia may preferably be obtained by polymerizing N-vinyl-2-pyrrolidone using hydrogen peroxide as a polymerization initiator in a presence of a metal catalyst using ammonia as a promoter in an aqueous medium.

The third process comprises polymerizing N-vinyl-2-pyrrrolidone using hydrogen peroxide as a polymerization initiator in a presence of a metal catalyst using ammonia as a promoter in an aqueous medium.

According to the present invention, high-quality polyvinylpyrrolidone can be obtained as a composition in a form of a solid preparation or an aqueous solution in a simple and easy manner. When the composition is in a form of a solid preparation, it may cause substantially no formation of insoluble matter and gelled matter, even if dissolved in an aqueous medium, so that it has excellent solubility and causes less coloration. Moreover, when the composition is in a form of an aqueous solution, it may cause substantially no formation of insoluble matter, even if heat dried, so that it has excellent heat resistance and low hue values.

The polyvinylpyrrolidone composition of the present invention can be used by itself as a raw material of an additive in a wide range of areas for applications such cosmetics, intermediates for drugs and agricultural chemicals, food additives, photosensitive electronic materials, and adhesion providing agents, or for various special industrial applications (e.g., the production of hollow fiber membranes). The polyvinylpyrrolidone composition of the present invention is particularly preferably suitable for applications requiring less insoluble matter formation and less coloration because it has excellent solubility and causes less coloration when it is in a form of a solid preparation or because it has excellent heat resistance and a low hue value when it is in a form of an aqueous solution.

### DETAILED DESCRIPTION OF THE INVENTION

<<Polyvinylpyrrolidone Composition>>

The polyvinylpyrrolidone composition of the present invention is characterized in that a solid preparation or an aqueous solution of polyvinylpyrrolidone comprises ammonia and a secondary amine. The solid preparation may preferably be a polyvinylpyrrolidone powder, but may also be in shapes such as particular, granular, spherical, aggregated, and amorphous shapes. The size of particles or the like forming a solid preparation, although it is not particularly limited, may appropriately be adjusted according to the applications of the solid preparation.

Polyvinylpyrrolidone is the homopolymer of N-vinyl-2-pyrrolidone, the molecular weight of which, although it is not particularly limited, may appropriately be adjusted according to the applications of polyvinylpyrrolidone, but may preferably have a lower limit of 10 and an upper limit of 60, more preferably of 50, when the molecular weight is expressed by K values which are determined by the Fikentscher method. The K values which are determined by the Fikentscher method are those which are measured by the method described below in Examples. The polyvinylpyrrolidone composition of the present invention may any other ingredient than polyvinylpyrrolidone, ammonia, and a secondary amine, so long as the effects of the present invention are not deteriorated. When the composition is in a form of a solid preparation, it may preferably be composed substantially of polyvinylpyrrolidone, except that it contains ammonia and a secondary amine. When the composition is in a form of an aqueous solution, the concentration of polyvinylpyrrolidone may preferably have a lower limit of 30% by mass, more preferably of 40% by mass, and an upper limit of 60% by mass, more preferably of 55% by mass.

Polyvinylpyrrolidone can be obtained by polymerizing N-vinyl-2-pyrrolidone, the method for which may be any of the methods well known in the art, but may preferably, from a viewpoint of suppressing coloration, be a method in which polymerization is carried out in a presence of a metal catalyst using ammonia as a promoter in an aqueous medium. The use of such a method results in that polyvinylpyrrolidone can be obtained in a form of an aqueous solution and inevitably contains ammonia. When the composition is in a form of an aqueous solution, the amount of ammonia contained therein may preferably have a lower limit of 50 ppm and an upper limit of 4,000 ppm, more preferably of 3,000 ppm, and still more preferably of 2,000 ppm. When the composition is in a form of a solid preparation, it can be obtained by heat drying a polyvinylpyrrolidone aqueous solution, so that the amount of ammonia contained therein may vary depending on the method of heat drying. For example, when the spray dryer drying method is used, the amount of ammonia contained therein may preferably have a lower limit of 50 ppm and an upper limit of 500 ppm, more preferably of 300 ppm. When a drying method of the hot face adhesion type, such as the drum dryer drying method, is used, the amount of ammonia contained therein may preferably have a lower limit of 50 ppm and an upper limit of 3,000 ppm, more preferably of 500 ppm, and still more preferably of 100 ppm.

In the present invention, the term “secondary amine” refers to a compound obtained by replacing two hydrogen atoms of ammonia with two substituted or unsubstituted hydrocarbon groups (including the case where these hydrocarbon groups are combined together, as described below, to form a nitrogen-containing heterocyclic ring). The hydrocarbon groups may be the same or different from each other. The hydrocarbon groups may independently be selected from aliphatic hydrocarbon groups, alicyclic hydrocarbon groups, and aromatic hydrocarbon groups; or may be combined together with the adjacent nitrogen atom, or in some cases, with any other hetero atom(s) selected from nitrogen, oxygen, and sulfur atoms, to form a nitrogen-containing heterocyclic ring. The secondary amine may be used in a form of a crystalline salt, by reaction with an acid, such as a hydrochloride salt, a hydrogen bromide salt, a sulfate salt, or a carbonate salt.

Examples of the aliphatic hydrocarbon group may include alkyl groups having 1 to 4 carbon atoms and alkenyl groups having 2 or 3 carbon atoms, specific examples of which may include a methyl group, en ethyl group, a propyl group, an isopropyl group, a butyl group, an isobutyl group, a sec-butyl group, a tert-butyl group, a vinyl group, an allyl group, and an isopropenyl group.

Examples of the alicyclic hydrocarbon group may include cycloalkyl groups having 5 or 6 carbon atoms, specific examples of which may include a cyclopentyl group and a cyclohexyl group.

Examples of the aromatic hydrocarbon group may include aryl groups having 6 to 8 carbon atoms and aralkyl groups having 7 or 8 carbon atom, specific examples of which may include a phenyl group, a tolyl group, a xylyl group, a benzyl group, and a phenetyl group.

Examples of the nitrogen-containing heterocyclic ring may include heterocyclic rings containing 1 or 2 nitrogen atoms, specific examples of which may include a pyrrolidine ring, a piperidine ring, a piperazine ring, a morpholine ring, and a thiomorpholine ring.

When the hydrocarbon group has a substituent group, examples of the substituent group may include hydrocarbon groups (e.g., CH₃—, CH₃CH₂—, C₆H₅—), halogen groups (e.g., F—, Cl—, Br—, I—), a hydroxy group (HO—), a carboxy group (—COOH), an ester group (e.g., —COOCH₃, —COOC₂H₅, CH₃COO—, C₆H₅COO—), an acryl group (e.g., —CHO, CH₃CO—, C₆H₅CO—), a sulfanyl group (HS—), a sulfo group (—SO₃H), a sulfamoyl group (H₂N—SO₂—), an amino group (H₂N—), a cyano group (—CN), and a nitro group (—NO₂) The hydrocarbon group may have any one of these substituent group or two or more kinds of these substituent groups in combination.

Specific examples of the secondary amine may include aliphatic secondary amines such as dimethylamine, diethylamine, dipropylamine, diisopropylamine, N-methylethylamine, N-methylpropylamine, N-methylisopropylamine, N-methylbutylamine, N-methylisobutylamine, N-methylcyclohexylamine, N-ethylpropylamine, N-ethylisopropylamine, N-ethylbutylamine, N-ethylisobutylamine, N-ethylcyclohexylamine, N-methylvinylamine, and N-methylallylamine; aliphatic diamines and triamines such as N-methylethylenediamine, N-ethylethylenediamine, N,N′-dimethylethylenediamine, N,N′-diethylethylenediamine, N-methyltrimethylenediamine, N-ethyltrimethylenediamine, N,N′-dimethyltrimethylenediamine, N,N′-diethyltrimethylenediamine, diethylenetriamine, and dipropylenetriamine; aromatic amines such as N-methylbenzylamine, N-ethylbenzylamine, N-methylphenetylamine, and N-ethylphenetylamine; monoalkanolamines such as N-methylethanolamine, N-ethylethanolamine, N-propylethanolamine, N-isopropylethanolamine, N-butylethanolamine, and N-isobutylethanolamine; dialkanolamines such as diethanolamine, dipropanolamine, diisopropanolamine, and dibutanolamine; and cyclic amines such as pyrrolidine, piperidine, piperazine, N-methylpiperazine, N-ethylpiperazine, morpholine, and thiomorpholine These secondary amines may be used alone, or two or more kinds of these secondary amines may also be used in combination. In these secondary amines, preferred are dialkanolamines and dialkylamiens, and more preferred are dialkanolamines, and particularly preferred is diethanolamine.

The secondary amine may be incorporated into the composition, by the use of a promoter in the polymerization of N-vinyl-2-pyrrolidone, or by the addition to a solid preparation or an aqueous solution of polyvinylpyrrolidone. In both cases, the amount of secondary amine contained in the polyvinylpyrrolidone composition finally obtained may preferably have a lower limit of 500 ppm, more preferably of 1,000 ppm, and an upper limit of 5,000 ppm, more preferably of 4,000 ppm.

The polyvinylpyrrolidone composition of the present invention, when it is in a form of a solid preparation, may cause substantially no formation of insoluble matter or gelled matter, even if dissolved in an aqueous medium, so that it has excellent solubility and causes less coloration. Moreover, the polyvinylpyrrolidone composition of the present invention, when it is in a form of an aqueous solution, may cause substantially no formation of insoluble matter, even if heat dried, so that it has excellent heat resistance and low hue values. However, 500 ppm or smaller of insoluble matter may remain in some cases, when the composition in a form of a solid preparation is dissolved in an aqueous medium and the solution is filtered, or when the composition in a form of an aqueous solution is heat dried to obtain a solid preparation, which is then dissolved again in an aqueous medium and the solution is filtered. Even such cases may be included in the scope of the present invention, assuming that the composition may cause substantially no formation of insoluble matter.

The polyvinylpyrrolidone composition of the present invention may be used for applications such as cosmetics, intermediates of drugs and agricultural chemicals, food additives, photosensitive electronic materials, and adhesion providing agents, or for various special industrial applications (e.g., the production of hollow fiber membranes), in its own form, or when it is in a form of an aqueous solution, after diluted or concentrated, or after heat dried to be changed into a form of a solid preparation.

<<Process for Producing Polyvinylpyrrolidone Composition>>

The polyvinylpyrrolidone composition in a form of a solid preparation can be obtained by heat drying a polyvinylpyrrolidone aqueous solution containing ammonia and a secondary amine. The polyvinylpyrrolidone aqueous solution may preferably be obtained by polymerizing N-vinyl-2-pyrrolidone using hydrogen peroxide as a polymerization initiator in a presence of a metal catalyst using ammonia and a secondary amine as promoters in an aqueous medium; or by adding a secondary amine to an aqueous solution containing polyvinylpyrrolidone and ammonia. The aqueous solution containing polyvinylpyrrolidone and ammonia may preferably be obtained by polymerizing N-vinyl-2-pyrrolidone using hydrogen peroxide as a polymerization initiator in a presence of a metal catalyst using ammonia as a promoter in an aqueous medium.

The polyvinylpyrrolidone composition in a form of an aqueous solution can be obtained by, for example, adding a secondary amine to an aqueous solution containing polyvinylpyrrolidone and ammonia. The aqueous solution containing polyvinylpyrrolidone and ammonia may preferably be obtained by polymerizing N-vinyl-2-pyrrolidone using hydrogen peroxide as a polymerization initiator in a presence of a metal catalyst using ammonia as a promoter in an aqueous medium.

Alternatively, the polyvinylpyrrolidone composition in a form of an aqueous solution can also be obtained by polymerizing N-vinyl-2-pyrrolidone using hydrogen peroxide as a polymerization initiator in a presence of a metal catalyst using ammonia and a secondary amine as promoters in an aqueous medium

When the polyvinylpyrrolidone composition in a form of a solid preparation, the method of heat drying a polyvinylpyrrolidone aqueous solution containing ammonia and a secondary amine, although it is not particularly limited, may be any of the methods well known in the art, but may preferably include the spray dryer drying method, the drum dryer drying method, the fluidized bed drying method, and the belt drying method. These heat drying methods may be used alone, or two or more kinds of these heat drying methods may also be used in combination. In these heat drying methods, preferred are the spray dryer drying method and the drum dryer drying method. The temperature and time of heat drying, although they are not particularly limited, may appropriately be adjusted according to the method of heat drying used.

When the polyvinylpyrrolidone composition in a form of a solid preparation or an aqueous solution is produced, the amount of secondary amine to be added to an aqueous solution containing polyvinylpyrrolidone and ammonia, although it is not particularly limited, may appropriately be adjusted according to the amount of secondary amine contained in the polyvinylpyrrolidone composition finally obtained. The amount of secondary amine contained in the polyvinylpyrrolidone composition is as described above in the explanation of the polyvinylpyrrolidone composition.

When the polyvinylpyrrolidone composition in a form of a solid preparation or an aqueous solution, the polymerization of N-vinyl-2-pyrrolidone may preferably be carried out by radical polymerization using hydrogen peroxide as a polymerization initiator in a presence of a metal catalyst and an ammonia promoter in an aqueous medium. The procedure, although it is not particularly limited, may be any of the procedures well known in the art. For example, the polymerization can be carried out by successively adding N-vinyl-2-pyrrolidone, a promoter, and a polymerization initiator to an aqueous medium containing a metal catalyst. Alternatively, the polymerization can also be carried out by successively adding a metal catalyst, a promoter, and a polymerization initiator to an aqueous medium containing N-vinyl-2-pyrrolidone. The polymerization temperature may preferably have a lower limit of 50° C. and an upper limit of 100° C. The polymerization time, although it is not particularly limited, may appropriately be adjusted according to the desired molecular weight of polyvinylpyrrolidone.

The metal catalyst is not particularly limited, so long as it is any of the metal catalysts well known in the art and used in the polymerization of N-vinyl-2-pyrrolidone. Specific examples of the metal catalyst may include heavy metal salts such as copper (II) sulfate, copper (II) chloride, and copper (II) acetate. The amount of metal catalyst to be added, although it is not particularly limited, may appropriately be adjusted according to the amount of N-vinyl-2-pyrrolidone charged, but may preferably have a lower limit of 50 ppb and an upper limit of 400 ppb, by mass ratio to N-vinyl-2-pyrrolidone.

Ammonia to be used as a promoter may be added in its own form or as an aqueous solution. The amount of ammonia to be added, although it is not particularly limited, may appropriately be adjusted according to the amount of ammonia contained in the polyvinylpyrrolidone composition finally obtained. The amount of ammonia contained in the polyvinylpyrrolidone composition is as described above in the explanation of the polyvinylpyrrolidone composition.

When a secondary amine is used, in addition to ammonia, as a promoter, the secondary amine may be added in its own form or as an aqueous solution. For the secondary amine to be used as a promoter, there can be used those which are recited in the explanation of the polyvinylpyrrolidone composition. The amount of secondary amine to be added, although it is not particularly limited, may appropriately be adjusted according to the amount of secondary amine contained in the polyvinylpyrrolidone composition finally obtained. The amount of secondary amine contained in the polyvinylpyrrolidone composition is as described above in the explanation of the polyvinylpyrrolidone composition.

When ammonia is used as a promoter, or when ammonia and a secondary amine are used as promoters, a primary amine and/or a tertiary amine may be used as additional promoters. The polyvinylpyrrolidone aqueous solution obtained by the only use of a primary amine, a secondary amine, or a tertiary amine may have an influence on its quality because it may cause the coloration of polyvinylpyrrolidone and the formation of insoluble matter when heat dried. However, the combined used of these amines and ammonia can suppress the coloration of polyvinylpyrrolidone. When a primary amine and/or a tertiary amine are used in combination with ammonia, the amount of each of these amines to be added, although it is not particularly limited, so long as the effects of the present invention are not deteriorated, may appropriately be adjusted according to the amount of ammonia to be added.

Hydrogen peroxide to be used as a polymerization initiator may be added in its own form or as an aqueous solution. The amount of hydrogen peroxide to be added, although it is not particularly limited, may appropriately be adjusted according to the amount of N-vinyl-2-pyrrolidone charged, but may preferably have a lower limit of 0.1% and an upper limit of 6%, by mass ratio to N-vinyl-2-pyrrolidone.

When N-vinyl-2-pyrrolidone is polymerized by the method as described above, polyvinylpyrrolidone can be obtained in a form of an aqueous solution. This polyvinylpyrrolidone aqueous solution, when a secondary amine is used, in addition to ammonia, as a promoter, may be used as a polyvinylpyrrolidone composition in a form of an aqueous solution, in its own form or by dilution or concentration, or may be converted into a polyvinylpyrrolidone composition in a form of a solid preparation by heat drying using any of the methods well known in the art. When only ammonia is used as a promoter, an aqueous solution containing polyvinylpyrrolidone and ammonia can be obtained, which may be used, after the addition of a secondary amine, as a polyvinylpyrrolidone composition in a form of an aqueous solution, in its own form or by dilution or concentration, or may be converted into a polyvinylpyrrolidone composition in a form of solid preparation by heat drying using any of the methods well known in the art.

According to the production process of the present invention, only a secondary amine is used at an appropriate stage, so that high quality polyvinylpyrrolidone can be produced as a composition in a form of solid preparation or an aqueous solution in a simple and easy manner.

### EXAMPLES

The present invention will be described below in detail by reference to Examples and Comparative Examples, but the present invention is not limited to these Examples. The present invention can be put into practice after appropriate modifications or variations within a range meeting the gists described above and later, all of which are included in the technical scope of the present invention. In the following Examples and Comparative Examples, unless otherwise indicated, the term “part(s)” means “part(s) by mass”.

First described are the methods of evaluation for polyvinylpyrrolidone compositions in a form of a solid preparation or an aqueous solution, which were obtained in Examples and Comparative Examples.

<K Value>

Each of the resulting polyvinylpyrrolidone aqueous solutions was diluted with water so that the dilution had a concentration of 1% by mass, and the dilution was measured for viscosity at 25° C. with a capillary viscometer. The K value was determined according to the Fikentscher's formula. A smaller value means that the polyvinylpyrrolidone has a smaller molecular weight.

<Hue (5% APHA)>

Each of the resulting polyvinylpyrrolidone aqueous solutions was diluted with water so that the dilution had a concentration of 5% by mass, and the dilution was measured for hue (5% APHA; also referred to as the Hazen color index) according to JIS-K3331. A smaller value means that the polyvinylpyrrolidone has a lower hue.

<Solubility>

First, 5 g of each of the resulting solid preparation of polyvinylpyrrolidone was further heated at 150° C. for 2 hours, to which 45 g of water was added to prepare a polyvinylpyrrolidone aqueous solution having a concentration of 10% by mass. The conditions of the aqueous solution was visually observed and evaluated as follows:

“∘”: excellent solubility with the formation of substantially no insoluble matter and gelled matter; and

“x”: poor solubility with the formation of insoluble matter and gelled matter.

<Coloration>

The polyvinylpyrrolidone aqueous solution obtained in the solubility test was diluted with water so that the dilution had a concentration of 5% by mass, and the dilution was measured for hue (5% APHA; also referred to as the Hazen color index) according to JIS-K3331. A smaller value means that the polyvinylpyrrolidone caused less coloration.

### Example 1

A reaction vessel was charged with 0.00023 parts of copper (II) sulfate and 430.8 parts of water, followed by heating up to 80° C. Then, while keeping the reaction vessel at 80° C., 450 parts of N-vinyl-2-pyrrolidone, 0.9 parts of 25% aqueous ammonia, 1.25 parts of diethanolamine, and 9 parts of 30% hydrogen peroxide were separately added dropwise to the reaction vessel over 180 minutes. After the completion of dropwise addition, 2.7 parts of 30% hydrogen peroxide was divided into 3 aliquots, and these aliquots were added to the reaction vessel at 1.5 hours intervals. After the third addition, the reaction vessel was kept at 80° C. for another 1 hour to obtain a polyvinylpyrrolidone aqueous solution. The resulting polyvinylpyrrolidone aqueous solution was measured for physical properties, and it was found that the concentration was 50% by mass; the K value was 29; the hue value (5% APHA) was 10; and the amount of remaining N-vinyl-2-pyrrolidone was not greater than 10 ppm, relative to polyvinylpyrrolidone. Then, 10 g of the resulting polyvinylpyrrolidone aqueous solution was heat dried at 150° C. for 1 hour to obtain 5 g of a solid preparation of polyvinylpyrrolidone. The resulting solid preparation of polyvinylpyrrolidone was evaluated for solubility and coloration. The results are shown in Table 1. In Table 1, “PVP” is an abbreviation of polyvinylpyrrolidone.

### Example 2

A reaction vessel was charged with 0.00023 parts of copper (II) sulfate and 430.8 parts of water, followed by heating up to 80° C. Then, while keeping the reaction vessel at 80° C., 450 parts of N-vinyl-2-pyrrolidone, 0.9 parts of 25% aqueous ammonia, and 9 parts of 30% hydrogen peroxide were separately added dropwise to the reaction vessel over 180 minutes. After the completion of dropwise addition, 2.7 parts of 30% hydrogen peroxide was divided into 3 aliquots, and these aliquots were added to the reaction vessel at 1.5 hours intervals. After the third addition, the reaction vessel was kept at 80° C. for another 1 hour, to which 1.25 parts of diethanolamine was then added to obtain a polyvinylpyrrolidone aqueous solution. The resulting polyvinylpyrrolidone aqueous solution was measured for physical properties, and it was found that the concentration was 50% by mass; the K value was 29; the hue value (5% APHA) was 10; and the amount of remaining N-vinyl-2-pyrrolidone was not greater than 10 ppm, relative to polyvinylpyrrolidone. Then, 10 g of the resulting polyvinylpyrrolidone aqueous solution was heat dried at 150° C. for 3 hours to obtain 5 g of a solid preparation of polyvinylpyrrolidone. The resulting solid preparation of polyvinylpyrrolidone was evaluated for solubility and coloration. The results are shown in Table 1.

### Comparative Example 1

A reaction vessel was charged with 0.00023 parts of copper (II) sulfate and 431.9 parts of water, followed by heating up to 80° C. Then, while keeping the reaction vessel at 80° C., 450 parts of N-vinyl-2-pyrrolidone, 0.9 parts of 25% ammonia solution, and 9 parts of 30% hydrogen peroxide were separately added dropwise to the reaction vessel over 180 minutes. After the completion of dropwise addition, 2.7 parts of 30% hydrogen peroxide was divided into 3 aliquots, and these aliquots were added to the reaction vessel at 1.5 hours intervals. After the third addition, the reaction vessel was kept at 80° C. for another 1 hour to obtain a polyvinylpyrrolidone aqueous solution. The resulting polyvinylpyrrolidone aqueous solution was measured for physical properties, and it was found that the concentration was 50% by mass; the K value was 29; the hue value (5% APHA) was 5; and the amount of remaining N-vinyl-2-pyrrolidone was not greater than 10 ppm, relative to polyvinylpyrrolidone. Then, 10 g of the resulting polyvinylpyrrolidone aqueous solution was heat dried at 150° C. for 3 hours to obtain 5 g of a solid preparation of polyvinylpyrrolidone. The resulting solid preparation of polyvinylpyrrolidone was evaluated for solubility and coloration. The results are shown in Table 1.

### Comparative Example 2

A reaction vessel was charged with 0.00023 parts of copper (II) sulfate and 430.2 parts of water, followed by heating up to 80° C. Then, while keeping the reaction vessel at 80° C., 450 parts of N-vinyl-2-pyrrolidone, 2.69 parts of diethanolamine, and 9 parts of 30% hydrogen peroxide were separately added dropwise to the reaction vessel over 180 minutes. After the completion of dropwise addition, 2.7 parts of 30% hydrogen peroxide was divided into 3 aliquots, and these aliquots were added to the reaction vessel at 1.5 hours intervals. After the third addition, the reaction vessel was kept at 80° C. for another 1 hour to obtain a polyvinylpyrrolidone aqueous solution. The resulting polyvinylpyrrolidone aqueous solution was measured for physical properties, and it was found that the concentration was 50% by mass; the K value was 28; the hue value (5% APHA) was 30; and the amount of remaining N-vinyl-2-pyrrolidone was not greater than 10 ppm, relative to polyvinylpyrrolidone. Then, 10 g of the resulting polyvinylpyrrolidone aqueous solution was heat dried at 150° C. for 3 hours to obtain 5 g of a solid preparation of polyvinylpyrrolidone. The resulting solid preparation of polyvinylpyrrolidone was evaluated for solubility and coloration. The results are shown in Table 1.

### Comparative Example 3

A reaction vessel was charged with 0.00023 parts of copper (II) sulfate and 430.1 parts of water, followed by heating up to 80° C. Then, while keeping the reaction vessel at 80° C., 450 parts of N-vinyl-2-pyrrolidone, 0.9 parts of 25% ammonia solution, 1.92 parts of triethanolamine, and 9 parts of 30% hydrogen peroxide were separately added dropwise to the reaction vessel over 180 minutes. After the completion of dropwise addition, 2.7 parts of 30% hydrogen peroxide was divided into 3 aliquots, and these aliquots were added to the reaction vessel at 1.5 hours intervals. After the third addition, the reaction vessel was kept at 80° C. for another 1 hour to obtain a polyvinylpyrrolidone aqueous solution. The resulting polyvinylpyrrolidone aqueous solution was measured for physical properties, and it was found that the concentration was 50% by mass; the K value was 29; the hue value (5% APHA) was 30; and the amount of remaining N-vinyl-2-pyrrolidone was not greater than 10 ppm, relative to polyvinylpyrrolidone. Then, 10 g of the resulting polyvinylpyrrolidone aqueous solution was heat dried at 150° C. for 3 hours to obtain 5 g of a solid preparation of polyvinylpyrrolidone. The resulting solid preparation of polyvinylpyrrolidone was evaluated for solubility and coloration. The results are shown in Table 1.

### Comparative Example 4

A reaction vessel was charged with 0.00023 parts of copper (II) sulfate and 431.3 parts of water, followed by heating up to 80° C. Then, while keeping the reaction vessel at 80° C., 450 parts of N-vinyl-2-pyrrolidone, 0.9 parts of 25% ammonia solution, 0.74 parts of monoethanolamine, and 9 parts of 30% hydrogen peroxide were separately added dropwise to the reaction vessel over 180 minutes. After the completion of dropwise addition, 2.7 parts of 30% hydrogen peroxide was divided into 3 aliquots, and these aliquots were added to the reaction vessel at 1.5 hours intervals. After the third addition, the reaction vessel was kept at 80° C. for another 1 hour to obtain a polyvinylpyrrolidone aqueous solution. The resulting polyvinylpyrrolidone aqueous solution was measured for physical properties, and it was found that the concentration was 50% by mass; the K value was 29; the hue (5% APHA) was 30; and the amount of remaining N-vinyl-2-pyrrolidone was not greater than 10 ppm, relative to polyvinylpyrrolidone. Then, 10 g of the resulting polyvinylpyrrolidone aqueous solution was heat dried at 150° C. for 3 hours to obtain 5 g of a solid preparation of polyvinylpyrrolidone. The resulting solid preparation of polyvinylpyrrolidone was evaluated for solubility and coloration. The results are shown in Table 1.

TABLE 1

| | | | Physical Properties | | | |
|---|---|---|---|---|---|---|
| | | | of PVP aqueous | | Evaluation of | |
| | Promoter(s) | Secondary amine | solutions | | PVP solid preparations | |
| | used in PVP | added after PVP | | Hue value | | Coloration |
| | preparation | preparation | K value | (5% APHA) | Solubility | (5% APHA) |
| Example 1 | Ammonia + diethanolamine | — | 29 | 10 | ◯ | 160 |
| Example 2 | Ammonia | Diethanolamine | 29 | 10 | ◯ | 160 |
| Comp. Ex. 1 | Ammonia | — | 29 | 5 | X | —* |
| Comp. Ex. 2 | Diethanolamine | — | 28 | 30 | ◯ | 240 |
| Comp. Ex. 3 | Ammonia + triethanolamine | — | 29 | 30 | X | —* |
| Comp. Ex. 4 | Ammonia + monoethanolamine | — | 29 | 30 | X | —* |

*These samples cannot be evaluated because they caused gelation after dissolved in water again.

As shown in Table 1, the polyvinylpyrrolidone composition of Example 1 obtained by using ammonia and diethanolamine (secondary amine) as promoters in the preparation of polyvinylpyrrolidone, and the polyvinylpyrrolidone preparation of Example 2 obtained by using ammonia as a promoter in the preparation of polyvinylpyrrolidone and then adding diethanolamine (secondary amine) after the preparation of polyvinylpyrrolidone, had excellent heat resistance because they caused substantially no formation of insoluble matter, even if heat dried, and had relatively low hue values, when they were in a form of an aqueous solution; and had excellent solubility because they caused substantially no formation of insoluble matter and gelled matter, even if dissolved in an aqueous medium, and caused less coloration, when they were in a form of a solid preparation.

In contrast, the polyvinylpyrrolidone composition of Comparative Example 1 obtained by using ammonia as a promoter in the preparation of polyvinylpyrrolidone and adding no diethanolamine after the preparation of polyvinylpyrrolidone, it had a relatively low hue value, but had poor heat resistance because it caused the formation of insoluble matter or gelled matter, if heat dried, when it is in a form of an aqueous solution; and had poor solubility because it caused gelation, if dissolved in an aqueous medium, when it is in a form of a solid preparation. Moreover, the polyvinylpyrrolidone composition of Comparative Example 2 obtained by using diethanolamine (secondary amine) as a promoter in the preparation of polyvinylpyrrolidone, had excellent heat resistance because it caused no formation of insoluble matter, if heat dried, but had a relatively high hue value, when it is in a form of an aqueous solution; and had excellent solubility because it caused substantially no formation of insoluble matter or gelled matter, even if dissolved in an aqueous medium, but was relatively more colored, when it is in a form of a solid preparation. Further, the polyvinylpyrrolidone composition of Comparative Example 3 obtained by using ammonia and triethanolamine (tertiary amine) as promoters in the preparation of polyvinylpyrrolidone, and the polyvinylpyrrolidone composition of Comparative Example 4 obtained by using ammonia and monoethanolamine (primary amine) as promoters in the preparation of polyvinylpyrrolidone, had poor heat resistance because they caused the formation of insoluble matter or gelled matter, if heat dried, and were relatively more colored, when they were in a form of an aqueous solution; and poor solubility because they caused gelation, if dissolved in an aqueous medium, when they were in a form of a solid preparation.

From these facts, it can be seen that polyvinylpyrrolidone compositions can attain, if they contain both ammonia and a secondary amine, the effects of having excellent heat resistance and having a low hue value, when they are in a form of an aqueous solution, and the effects of having excellent solubility and causing less coloration, when they are in a form of a solid preparation, whereas they cannot attain such effects, if either one of ammonia or a secondary amine is used, or if ammonia and a primary amine are used in combination or ammonia and a tertiary amine are used in combination.

1. A polyvinylpyrrolidone composition in a form of a solid preparation or an aqueous solution, the composition comprising polyvinylpyrrolidone, ammonia, and a secondary amine.

2. The polyvinylpyrrolidone composition according to claim 1, wherein the secondary amine is selected from the group consisting of dialkanolamines and dialkylamines.

3. The polyvinylpyrrolidone composition according to claim 2, wherein the secondary amine is diethanolamine.

4. A process for producing a polyvinylpyrrolidone composition according to claim 1, the process comprising heat drying a polyvinylpyrrolidone aqueous solution containing ammonia and a secondary amine to obtain the polyvinylpyrrolidone composition in a form of a solid preparation.

5. The process according to claim 4, wherein the polyvinylpyrrolidone aqueous solution is obtained by polymerizing N-vinyl-2-pyrrolidone using hydrogen peroxide as a polymerization initiator in a presence of a metal catalyst using ammonia and the secondary amine as promoters in an aqueous medium.

6. The process according to claim 4, wherein the polyvinylpyrrolidone aqueous solution is obtained by adding the secondary amine to an aqueous solution containing polyvinylpyrrolidone and ammonia.

7. The process according to claim 6, wherein the aqueous solution containing polyvinylpyrrolidone and ammonia is obtained by polymerizing N-vinyl-2-pyrrolidone using hydrogen peroxide as a polymerization initiator in a presence of a metal catalyst using ammonia as a promoter in an aqueous medium.

8. A process for producing a polyvinylpyrrolidone composition according to claim 1, the process comprising adding a secondary amine to an aqueous solution containing polyvinylpyrrolidone and ammonia to obtain the polyvinylpyrrolidone composition in a form of an aqueous solution.

9. The process according to claim 8, wherein the aqueous solution containing polyvinylpyrrolidone and ammonia is obtained by polymerizing N-vinyl-2-pyrrolidone using hydrogen peroxide as a polymerization initiator in a presence of a metal catalyst using ammonia as a promoter in an aqueous medium.

10. A process for producing a polyvinylpyrrolidone composition according to claim 1, the process comprising polymerizing N-vinyl-2-pyrrrolidone using hydrogen peroxide as a polymerization initiator in a presence of a metal catalyst using ammonia as a promoter in an aqueous medium.
